(19) United States
(12) Patent Application Publication
(10) Pub. No.: US 2024/0390359 A1
(43) Pub. Date: Nov. 28, 2024

(54) METHODS FOR THE TREATMENT AND PREVENTION OF NON-VIRAL TICK-BORNE DISEASES AND SYMPTOMS THEREOF

(71) Applicants: 60 DEGREES PHARMACEUTICALS LLC, Washington, DC (US); Tufts Medical Center, Inc., Boston, MA (US)

(72) Inventors: Geoffrey S. DOW, Washington, DC (US); Bryan L. Smith, Chevy Chase, MD (US); Edouard Vannier, Boston, MA (US)

(73) Assignees: 60 DEGREES PHARMACEUTICALS LLC, Washington, DC (US); Tufts Medical Center, Inc., Boston, MA (US)

(21) Appl. No.: 18/640,657

(22) Filed: Apr. 19, 2024

Related U.S. Application Data
(60) Provisional application No. 63/461,060, filed on Apr. 21, 2023.

(51) Int. Cl. A61K 31/4706; A61K 31/122; A61K 31/65; A61K 31/7052; A61P 33/02

(52) U.S. Cl. CPC A61K 31/4706; A61K 31/122; A61K 31/65; A61K 31/7052; A61P 33/02

(57) ABSTRACT

Methods and compositions for treating or preventing non-viral tick-borne diseases and symptoms thereof by administering a long half-life 8-aminoquinoline, such as tafenoquine, are disclosed. Kits including a means for testing for a non-viral tick-borne disease and/or symptoms thereof and a long half-life 8-aminoquinoline, such as tafenoquine, are disclosed.

### BACKGROUND OF THE INVENTION

8-aminoquinolines have the capacity to inhibit or kill intracellular and extracellular pathogens via induction of oxidative stress, and thereby to prevent or treat diseases caused by such pathogens, provided that (i) the pathogen is innately susceptible to tafenoquine or a hydrogen peroxide-like mechanism of action in the mammalian host, (ii) the 8-aminoquinoline has the appropriate substituents to impart a long half-life and therefore an infrequent dosing schedule, (iii) that they can be safely dosed at levels high enough to achieve the desired antimicrobial or antiparasitic effect for sufficiently long periods, and/or (iv) the dose selected for administration is appropriate to treat both immune and immunocompetent patients without dose adjustment. As outlined below, standard medical care and treatment options for human diseases caused by the transmission of certain micro-organisms by ticks are insufficient and could be improved upon through the use of a long half-life 8-aminoquinoline alone or in combination with other agents.

Ticks found throughout the United States include American dog ticks, blacklegged ticks, brown dog ticks, groundhog ticks, gulf coast ticks, lone star ticks, Rocky Mountain wood ticks, soft ticks, and western blacklegged ticks. Some ticks carry pathogens such as Anaplasma spp, Babesia spp, Borrelia spp, Ehrlichia spp, Rickettsia spp, and Francisella spp, which can cause human disease, including African tick bite fever, anaplasmosis (formerly known as Human Granulocytic Ehrlichiosis (“HGE”)), babesiosis, ehrlichiosis, Lyme disease, Mediterranean spotted fever (also known as boutonneuse fever), Rickettsia parkeri rickettsiosis, Rocky Mountain spotted fever, Southern tick-associated rash illness, tickborne relapsing fever, tularemia, and 364D rickettsiosis (a relatively new disease found in California, U.S.A.), amongst others. The infecting organisms (with the exception of Borrelia spp) all have obligate intracellular lifecycles in the mammalian host and all (with the exception of Babesia spp) are gram-negative spirochetes. From 2004 to 2019, the total reported cases of tickborne diseases in the United States has gone from 20,000 to as high as 60,000. Estimates for unreported cases range from 9 to 30 times higher, i.e., from 180,000 to 1,800,000. The diseases caused by ticks range in severity.

### Babesiosis

Babesiosis, caused by various spp of Babesia, has an incubation period of 1-9 or more weeks and signs and symptoms of babesiosis include fever, chills, sweat, malaise, fatigue, myalgia, arthralgia, headache, gastrointestinal symptoms (such as anorexia and nausea, and less commonly abdominal pain and vomiting), and dark urine. Less common signs and symptoms include cough, sore throat, emotional lability, depression, photophobia, conjunctival injection, mild splenomegaly, mild hepatomegaly, and jaundice. General laboratory findings for babesiosis include decreased hematocrit, due to hemolytic anemia, thrombocytopenia, elevated serum creatinine and blood urea nitrogen values, and mildly elevated hepatic transaminase values.

Conventional treatment for babesiosis is for at least 7-10 days with a combination of two medicines, typically either atovaquone plus azithromycin or clindamycin plus quinine. These regimens generally cure most immunocompetent patients, but do not prevent a high rate (>30%) of complications amongst hospitalized patients and additional rounds of chemotherapy are required in approximately 10% and 25% of immunocompetent and immunocompromised hospitalized patients, respectively. Amongst the general hospitalized population the mortality rate is 1-2%, but may be substantially higher in immunocompromised individuals. In contrast, malaria, a disease also caused by protozoan parasites that invade red blood cells and can cause anemia and severe organ failure, has effective 3-day oral and iv treatments, and a mortality rate of 0.35% in the United States (Mace et al. 2021).

### Lyme Disease and Borellioses

Lyme disease and borrelioses are associated with infection by Borrelia spp, the most important of which is B. burgdorferi in the United States. The incubation period is 3-30 days and the signs and symptoms vary based upon stage. For the localized stage, signs and symptoms include erythema migrans (“EM”)-red, ring-like or homogenous expanding rash, flu-like symptoms (such as malaise, headache, fever, myalgia, and arthralgia), and lymphadenopathy. For the disseminated stage, signs and symptoms include multiple secondary annular rashes, flu-like symptoms, lymphadenopathy, rheumatologic manifestiations, cardiac manifestations, neurologic manifestations, conjunctivitis, keratitis, uveitis, mild hepatitis, and splenomegaly. General laboratory findings include elevated erythrocyte sedimentation rate, mildly elevated hepatic transaminases, microscopic hematuria or proteinuria, and in Lyme meningitis, cerebrospinal fluid typically shows lymphocytic pleocytosis, slightly elevated protein, and normal glucose. Post-exposure prophylaxis for Lyme disease is a single dose of 200 mg doxycycline within 72h of a tick-bite (Lantos et al. 2021). Erythema migrans is treated with 10-14 days of oral antibiotic therapy doxycycline, cefuroxime axetil, and amoxicillin with azithromycin for 5-10 days as the second line alternative (Lantos et al. 2021). Treatment of established infections and their complications is complicated and may involve administration of oral or intravenous antibiotics (including the previously mentioned and penicillin G) and corticosteroids depending on the setting (Lantos et al. 2021).

Lyme disease is not associated with a high mortality rate, and in a review of mortality records between 1993-2003 in the United States, only one case consistent with a clinical presentation of Lyme was observed (Kugeler et al. 2010). However, while treatment with standard antibiotics leads to successful clinical resolution in >80% of cases, Lyme disease may persist in the remainder of patients in syndrome with various manifestations referred to post-treatment Lyme disease syndrome (PTLDS, Cabello et al 2022). The causes of PTLDS are not precisely known (Cabello et al. 2022), but antimicrobial tolerance is one mechanism that has been proposed. Periods of transient intracellular localization of Borrelia organisms enabling evasion of immune defenses has also been proposed (Ma et al. 1991, Montgomery et al. 1993).

Another borreliosis is Borrelia miyamotoi disease, the incubation period of which is from days to weeks and signs and symptoms of Borrelia miyamotoi disease include fever, chills, fatigue, severe headache, arthralgia/myalgia, dizziness, confusion, vertigo, rash, dyspnea, nausea, abdominal pain, diarrhea, and anorexia. General laboratory findings for Borrelia miyamotoi disease include leukopenia, thrombocytopenia, and elevated hepatic transaminase values. There is not generally accepted treatment for Borrelia miyamotoi disease, antibiotics used to treat Lyme disease (at the same dosages) are reportedly effective.

### Tularemia

Tularemia, caused by Fancisella tularemia, is a highly infectious disease with a mortality rate as high as 30%. The incubation period is 3-5 days, but can be up to 21 days, and signs and symptoms include fever, chills, headache, malaise, fatigue, anorexia, myalgia, chest discomfort, cough, sore throat, vomiting, diarrhea, and abdominal pain. General laboratory findings include normal or elevated leukocyte count and sedimentation rate, thrombocytopenia, hyponatremia, elevated hepatic transaminases, elevated creatine phosphokinase, and myogloinuria and sterial pyuria may or may not be present. Conventional treatment for tularemia is with streptomycin, gentamicin, ciprofloxacin, and doxycycline.

### Other Tick-Borne Rickettsial Diseases (“TBRDs”)

TBRDs are caused by various obligate intracellular Rickettsia spp, the most important of which are Rickettsia rickettsii, Ehrlichia chaffeensis, and Anaplasma phagocytophilum in the United States (Chapman et al. 2006). Brief descriptions of some of the more important TBRDs are outlined below. What is generally true about all TBRDs is that their management may require hospitalization in up to 50% of cases, there is a high rate of in hospital complications, the standard of care is administration of tetracycline antibiotics, however they are most effective early infection when TBRDs are difficult to distinguish from other infectious diseases (Chapman et al. 2006). Although tetracyclines are considered the standard of care, mortality rates in the treated population may be as high as 5% in the case of rocky mountain spotted fever and 1-3% in the case of anaplasmosis and ehrlichosis. Given that diseases such as malaria rates have fatality rates as low as 0.35% (Mace et al. 2021) can be treated with three-day oral or IV medicines, it is clear that the standard of care for treatment of TBRDs is inadequate and patients are generally under-treated.

For anaplasmosis, the incubation period is generally 5-14 days and signs and symptoms of anaplasmosis include fever, chills, rigor, severe headache, malaise, myalgia, gastrointestinal symptoms (such as nausea, vomiting, diarrhea, and anorexia), and rash. General laboratory findings for anaplasmosis, typically observed during the first week of clinical disease, include mild anemia, thrombocytopenia, leukopenia (characterized by relative and absolute lymphopenia and a left shift), and mild to moderate elevations in hepatic transaminases. Visualization of morulae in the cytoplasm of granulocytes during examination of blood smears is highly suggestive of diagnosis.

For ehrlichiosis, the incubation period is 5-14 days and the signs and symptoms of ehrlichiosis include fever, chills, headache, malaise, muscle pain, gastrointestinal symptoms (such as nausea, vomiting, diarrhea, and anorexia), altered mental status, and rash. General laboratory findings for ehrlichiosis include thrombocytopenia, leukopenia (absolute), anemia (which generally occurs later in illness than thrombocytopenia or leukopenia), and mild to moderate elevations in hepatic transaminases.

For Rickettsia parkeri rickettsiosis, the incubation period is 2-10 days and signs and symptoms include fever, headache, rash (sparse maculopapular or papulavesicular eruptions on the truck and extremities), and muscle aches. General laboratory findings include mildly elevated hepatic transaminases, mild leukopenia, and mild thrombocytopenia.

For Rocky Mountain spotted fever disease, the incubation period is 3-12 days and the signs and symptoms include: in the first 1-4 days, high fever, severe headache, malaise, myalgia, edema around eyes and the back of hands, and gastrointestinal symptoms (including nausea, vomiting, and anorexia); at 5 days and beyond, altered mental status, coma, cerebral edema, respiratory compromise (pulmonary edema, ARDS), necrosis, and multiorgan system damage (CNS, renal failure). Rash typically appears 2-5 days after onset of symptoms. General laboratory findings include thrombocytopenia, elevated hepatic transaminases, and hyponatremia.

For tickborne relapsing fever, the incubation period is about 7 days, followed by recurring febrile episodes that last about 3 days and are separated by afebrile periods of about 7 days and the signs and symptoms include headache, myalgia, chills, nausea, vomiting, arthralgia, and facial palsy. General laboratory findings include normal to increased white blood cell count with a left shift, mildly increased serum bilirubin, mild to moderate thrombocytopenia, elevated erthrocyte sedimentation rate, slightly prolonged prothrombin time and partial thromboplastin time. Conventional treatment for tickborne relapsing fever is with tetracycline, erythromycin, and ceftriaxone.

There is an urgent need for new methods of treating and preventing non-viral tick-borne diseases, such as babesiosis, and symptoms thereof.

### SUMMARY OF THE INVENTION

The present invention concerns methods for treating or preventing a non-viral tick-borne disease, or a symptom thereof, in a human subject, said method comprising administering an effective amount of a long half-life 8-aminoquinoline to a subject in need thereof. In certain embodiments, the organism transmitted by a tick is caused by a Babesia, Rickettsia, Francisella, Anaplasma, Ehrlichia, or Borrelia spp and the disease caused may be one or more of, but is not limited to, the following: African tick bite fever, anaplasmosis, babesiosis, borreliosis, ehrlichiosis, Lyme disease, Mediterranean spotted fever, relapsing tick-fever, Rickettsia parkeri rickettsiosis, rickettsiosis, Rocky Mountain spotted fever, Southern tick-associated rash illness, tickborne relapsing fever, tularemia, and 364D rickettsiosis. In certain embodiments, the long half-life 8-aminoquinoline is capable of treating or preventing the tick-borne disease at a minimum single dose or first dose in a regimen of 50 mg. In certain embodiments, the long half-life 8-aminoquinoline is tafenoquine or a pharmaceutically acceptable salt thereof. In certain embodiments, administration is according to one of the following regimens: (a) a first dose of 50 mg followed by one additional dose of 50 mg within one week of the first dose; (b) a first dose of 100 mg followed by at least one and up to five additional doses within one week of the first dose; (c) a first dose of 150 mg followed by at least one and up to four additional doses within one week of the first dose; (d) a first dose of 200 mg followed by at least one and up to three additional doses within 15 days of the first dose; (e) a first dose of 300 mg followed by one additional dose of 300 mg within one week of the first dose; (f) a dose of 400 mg; (g) a loading dose of 600 mg taken over 1-5 days via administration of: (i) 6 100 mg doses, or (ii) 4 150 mg doses, or (iii) 3 200 mg doses; followed by a maintenance dose of 200 mg within 12 days of the first dose of the loading dose; and (h) a loading dose of 600 mg taken over 1-5 days via administration of: (i) 6 100 mg doses, or (ii) 4 150 mg doses or (iii) 3 200 mg doses; followed by a maintenance dose of 200 mg one week after completion of the loading dose followed by 200 mg once a week for up to 52 weeks. In certain embodiments, said subject in need thereof is: symptomatic ambulatory, symptomatic ambulatory with risk factors for disease progression, symptomatic ambulatory and immunocompromised, symptomatic hospitalized, symptomatic hospitalized with risk factors for disease progression, symptomatic hospitalized and immunocompromised, asymptomatic, asymptomatic without risk factors, asymptomatic with risk factors, asymptomatic at risk of tick bite, asymptomatic without risk factors at risk of tick bite, asymptomatic with risk factors at risk of tick bite, one without risk factors who has been bitten by a tick and is at risk of contracting said tick-borne disease, and one with risk factors who has been bitten by a tick and is at risk of contracting said tick-borne disease.

In certain embodiments, the invention concerns methods for treating or preventing a non-viral tick-borne disease, or a symptom thereof, in a human subject, said method comprising: (a) administering to said subject an effective amount of a compound of Formula (I), a pharmaceutically-acceptable salt thereof, or a pharmaceutical composition comprising a compound of Formula (I),

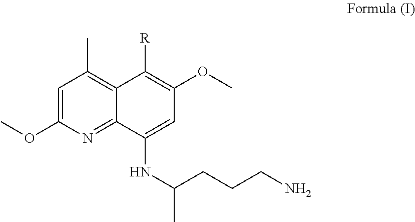

wherein R is any halogen-containing substituent of molecular weight≤205. In certain embodiments, the organism transmitted by a tick is caused by a Babesia, Rickettsia, Francisella, Anaplasma, Ehrlichia or Borrelia spp and the disease caused may be one or more of, but is not limited to the following: African tick bite fever, anaplasmosis, babesiosis, borreliosis, ehrlichiosis, lyme disease, Mediterranean spotted fever, relapsing tick-fever, Rickettsia parkeri rickettsiosis, rickettsiosis, Rocky Mountain spotted fever, Southern tick-associated rash illness, tickborne relapsing fever, tularemia, and 364D rickettsiosis. In certain embodiments, the long half-life 8-aminoquinoline is capable of treating or preventing the tick-borne disease at a minimum single dose or first dose in a regimen of 50 mg. In certain embodiments, the long half-life 8-aminoquinoline is tafenoquine or a pharmaceutically acceptable salt thereof. In certain embodiments, administration is accordingly to one of the following regimens: (a) a first dose of 50 mg followed by one additional dose of 50 mg within one week of the first dose; (b) a first dose of 100 mg followed by at least one and up to five additional doses within one week of the first dose; (c) a first dose of 150 mg followed by at least one and up to four additional doses within one week of the first dose; (d) a first dose of 200 mg followed by at least one and up to three additional doses within 15 days of the first dose; (e) a first dose of 300 mg followed by one additional dose of 300 mg within one week of the first dose; (f) a dose of 400 mg; (g) a loading dose of 600 mg taken over 1-5 days via administration of: (i) 6 100 mg doses, or (ii) 4 150 mg doses, or (iii) 3 200 mg doses; followed by a maintenance dose of 200 mg within 12 days of the first dose of the loading dose; and (h) a loading dose of 600 mg taken over 1-5 days via administration of: (i) 6 100 mg doses, or (ii) 4 150 mg doses or (iii) 3 200 mg doses; followed by a maintenance dose of 200 mg one week after completion of the loading dose followed by 200 mg once a week for up to 52 weeks. In certain embodiments, said subject in need thereof is: symptomatic ambulatory, symptomatic ambulatory with risk factors for disease progression, symptomatic ambulatory and immunocompromised, symptomatic hospitalized, symptomatic hospitalized with risk factors for disease progression, symptomatic hospitalized and immunocompromised, asymptomatic, asymptomatic without risk factors, asymptomatic with risk factors, asymptomatic at risk of tick bite, asymptomatic without risk factors at risk of tick bite, asymptomatic with risk factors at risk of tick bite, one without risk factors who has been bitten by a tick and is at risk of contracting said tick-borne disease, and one with risk factors who has been bitten by a tick and is at risk of contracting said tick-borne disease.

In certain embodiments, methods for treating or preventing a non-viral tick-borne disease, or a symptom thereof, in a human subject, said method comprising administering tafenoquine to a subject in need thereof are provided. In certain embodiments, the organism transmitted by a tick is caused by a Babesia, Rickettsia, Francisella, Anaplasma, Ehrlichia or Borellia spp and the disease caused may be one or more of, but is not limited to the following: African tick bite fever, anaplasmosis, babesiosis, borreliosis, ehrlichiosis, lyme disease, Mediterranean spotted fever, relapsing tick-fever, Rickettsia parkeri rickettsiosis, rickettsiosis, Rocky Mountain spotted fever, Southern tick-associated rash illness, tickborne relapsing fever, tularemia, and 364D rickettsiosis. In certain embodiments, the long half-life 8-aminoquinoline is capable of treating or preventing the tick-borne disease at a minimum single dose or first dose in a regimen of 50 mg. In certain embodiments, the long half-life 8-aminoquinoline is tafenoquine or a pharmaceutically acceptable salt thereof. In certain embodiments, administration is according to one of the following regimens: (a) a first dose of 50 mg followed by one additional dose of 50 mg within one week of the first dose; (b) a first dose of 100 mg followed by at least one and up to five additional doses within one week of the first dose; (c) a first dose of 150 mg followed by at least one and up to four additional doses within one week of the first dose; (d) a first dose of 200 mg followed by at least one and up to three additional doses within 15 days of the first dose; (e) a first dose of 300 mg followed by one additional dose of 300 mg within one week of the first dose; (f) a dose of 400 mg; (g) a loading dose of 600 mg taken over 1-5 days via administration of: (i) 6 100 mg doses, or (ii) 4 150 mg doses, or (iii) 3 200 mg doses; followed by a maintenance dose of 200 mg within 12 days of the first dose of the loading dose; and (h) a loading dose of 600 mg taken over 1-5 days via administration of: (i) 6 100 mg doses, or (ii) 4 150 mg doses or (iii) 3 200 mg doses; followed by a maintenance dose of 200 mg one week after completion of the loading dose followed by 200 mg once a week for up to 52 weeks. In certain embodiments, said subject in need thereof is: symptomatic ambulatory, symptomatic ambulatory with risk factors for disease progression, symptomatic ambulatory and immunocompromised, symptomatic hospitalized, symptomatic hospitalized with risk factors for disease progression, symptomatic hospitalized and immunocompromised, asymptomatic, asymptomatic without risk factors, asymptomatic with risk factors, asymptomatic at risk of tick bite, asymptomatic without risk factors at risk of tick bite, asymptomatic with risk factors at risk of tick bite, one without risk factors who has been bitten by a tick and is at risk of contracting said tick-borne disease, and one with risk factors who has been bitten by a tick and is at risk of contracting said tick-borne disease.

In certain embodiments, the methods of the invention further comprise administering a second and/or third agent, for example doxycycline, azithromycin-atovaquone, clindamycin-quinine, artesunate, artemether-lumefantrine, or any other agent[s] recommended by the IDSA or CDC, for treating or preventing a non-viral tick-borne disease.

For the methods of the invention, in certain embodiments risk factors for disease progression are selected from the group consisting of age >55 years, fatigue, nausea, diarrhea, duration of symptoms >7 days, lab abnormalities such as depressed/elevated white cell counts, elevated bilirubin and elevated createnine or any pre-existing condition or treatment for/or a condition that results in an immunocompromised state, and combinations thereof.

For the methods of the invention, in certain embodiments said immunocompromised state results from a condition selected from the group consisting of asplenia, hyposplenism, prior treatment with an immunosuppressive drug, pre-existing auto-immune disease or other condition known to suppress the immune system, and combinations thereof.

In certain embodiments, said causative agent is a species of Babesia or the disease is babesiosis.

In certain embodiments, said causative agent is a species of Borrelia or the disease is borreliosis or Lyme disease.

In certain embodiments, said causative agent is a species of Rickettsia or the disease is ricketsiosis.

In certain embodiments, said causative agent is a species of Anaplasma or Ehrlichia and the disease is anaplasmosis or ehrlichiosis.

In certain embodiments, said causative agent is a species of Francisella and the disease is tularemia.

In certain embodiments, administration is via sub-lingual and/or buccal and/or intravenous route(s).

In certain embodiments, administration is conducted according to the dosing regimen of Table 1 and/or according to any of the Examples.

In certain embodiments, no more than 11,000 mg of the long half-life 8-aminoquinoline is administered to said subject in a twelve-month period.

In certain embodiments, no more than 11,000 mg of a compound of Formula (I), a pharmaceutically-acceptable salt thereof, or a pharmaceutical composition comprising a compound of Formula (I), wherein R is any halogen-containing substituent of molecular weight≤205, is administered to said subject in a twelve-month period.

In certain embodiments, a kit comprising: (a) a means for testing for a non-viral tick-borne disease and/or symptoms thereof and/or glucose-6-dehydrogenase

(G6PD deficiency); (b) a compound of Formula (I), a pharmaceutically-acceptable salt thereof, or a pharmaceutical composition comprising a compound of Formula (I); and (c) instructions for use are provided, wherein R in Formula (I) is any halogen-containing substituent of molecular weight≤205.

In certain embodiments, a kit comprising: (a) a means for testing for a non-viral tick-borne disease and/or symptoms thereof and/or glucose-6-dehydrogenase

(G6PD deficiency); (b) a long half-life 8-aminoquinoline; and (c) instructions for use are provided.

In certain embodiments, a kit comprising: (a) a means for testing for a non-viral tick-borne disease and/or symptoms thereof and/or glucose-6-dehydrogenase (G6PD deficiency); (b) tafenoquine; and (c) instructions for use are provided. In certain embodiments, methods for pre-treating a non-viral tick-borne disease and/or symptoms thereof in a human subject are provided, said method comprising administering to said subject an effective amount of a compound of Formula (I), a pharmaceutically-acceptable salt thereof, or a pharmaceutical composition comprising a compound of Formula (I)

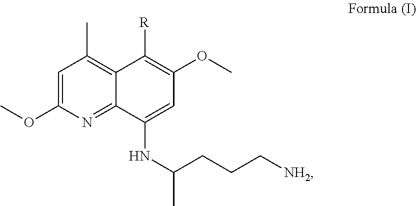

wherein R is any halogen-containing substituent of molecular weight≤205. In certain embodiments, said Formula (I) compound is tafenoquine or a salt thereof.

### Summary of Embodiments

The present invention is directed to methods for treating and/or preventing tick-borne diseases, or symptoms thereof, in human subjects. The methods comprise administering an effective amount of a long half-life 8-aminoquinoline to a subject in need of treatment for, or prevention of, tick-borne diseases. In another aspect, the invention is directed to use of a long half-life 8-aminoquinoline for treating or preventing a tick-borne disease, or a symptom thereof, in a human subject, comprising administering an effective amount of the long half-life 8-aminoquinoline to the human subject.

### Babesia Species

In some embodiments of the invention, the tick-borne disease is caused by a parasite of a Babesia species. In said embodiments, the tick-borne disease may be babesiosis. In some embodiments, the invention is directed to treating human subjects for tick-borne diseases caused by a parasite of a Babesia species, wherein the subject has been diagnosed as being infected with the parasite of the Babesia species prior to administering the long half-life 8-aminoquinoline to the subject. In some embodiments, e.g., where the subject is infected with the parasite of the Babesia species, said subject may be co-infected with a spirochete of a Borrelia species, or a parasite of a Borrelia species.

In some embodiments, the invention is directed to treating human subjects for tick-borne diseases caused by a parasite of a Babesia species, where the subject has been diagnosed with at least one of a Babesia infection and Babesia disease. In some embodiments, the diagnosis comprises a laboratory test that confirms a Babesia infection and/or Babesia disease.

In some embodiments, the subject being treating according to the invention is symptomatic ambulatory or symptomatic hospitalized. In some embodiments, the subject is symptomatic hospitalized.

In some embodiments, the invention is directed to preventing tick borne diseases in human subjects who have increased risk of exposure to, or who have had potential exposure to, a tick-borne pathogen comprising a parasite of the Babesia species.

### Non-Babesia Species

In some embodiments of the invention, the tick-borne disease is caused by a parasite of a non-babesia species. In said embodiments, the disease may be caused by a parasite of a Borrelia, Rickettsia, Francisella, Anaplasma, or Ehrlichia species. In some embodiments, the tick-borne disease may be selected from one or more of Lyme disease, borreliosis, African tick bite fever, anaplasmosis, ehrlichiosis, Mediterranean spotted fever, relapsing tick-fever, Rickettsia parkeri rickettsiosis, rickettsiosis, Rocky Mountain spotted fever, Southern tick-associated rash illness, tickborne relapsing fever, tularemia, and 364D rickettsiosis. In other embodiments, the tick-borne disease is caused by a parasite of a Borrelia species, and the long half-life 8-aminoquinoline is administered to the subject to prevent or reduce the severity of borreliosis and/or Lyme disease.

In some embodiments, e.g., comprising methods of treatment, or uses of the long half-life 8-aminoquinolineto treat a subject, the subject has been diagnosed as being infected with the parasite of a Borrelia, Rickettsia, Francisella, Anaplasma, or Ehrlichia species prior to administration of the long half-life 8-aminoquinoline. In other embodiments, the invention is directed to preventing tick borne diseases in human subjects who have increased risk of exposure to, or who have had potential exposure to, a tick-borne pathogen comprising a parasite of the Babesia species.

### Preventing Tick Disease Pre-Exposure and Post-Exposure

In embodiments of the invention directed to preventing tick-borne diseases (for example, caused by parasites of the babesia or non-babesia species), a human subject may be identified as in need of prevention where the subject is Identified as having an increased risk of exposure to a tick-borne pathogen. For example, in some embodiments, a subject at increased risk of exposure comprises a subject that is travelling to and/or conducting recreational or occupational activities in an area or environment associated with increased risk of tick bite. In some embodiments, the invention is directed to identifying the human subject in need of prevention. In some embodiments, the increased risk of tick bite comprises increased risk of a blacklegged tick bite.

In embodiments of the invention directed to preventing tick-borne diseases, a human subject may be identified as in need of prevention where the subject is Identified as having had potential exposure to a tick-borne pathogen, e.g., in some embodiments, wherein the tick-borne disease is caused by a parasite of a non-Babesia species, or in other embodiments, by a Babesia species. In some embodiments, the potential exposure may be a known or suspected tick bite from a tick known or suspected to be a blacklegged tick. In some embodiments, the potential exposure is a known or suspected tick bite. In other embodiments, the potential exposure is a known tick bite from a blacklegged tick.

### Compositions, Uses, and Administration

In each of the embodiments of the invention, the long half-life 8-aminoquinoline may be a compound capable of treating or preventing the tick-borne disease at a minimum single dose or first dose in a regimen of 50 mg. In some embodiments, the long half-life 8-aminoquinoline is a compound of Formula (I), a pharmaceutically-acceptable salt thereof,

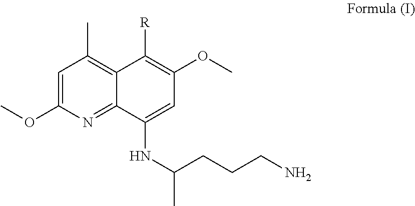

wherein R is any halogen-containing substituent of molecular weight≤205. In other embodiments, the long half-life 8-aminoquinoline may be a pharmaceutical composition comprising a compound of Formula (I) and a pharmaceutically-acceptable carrier. In other embodiments, the long half-life 8-aminoquinoline is tafenoquine or a pharmaceutically acceptable salt thereof. In other embodiments, the long half-life 8-aminoquinoline may be a pharmaceutical composition comprising tafenoquine and a pharmaceutically-acceptable carrier.

In each aspect of the invention, the long half-life 8-aminoquinoline may be administered to the subject (e.g., in need of treatment or prevention of tick-borne disease), according to one of the following regimens:
(a) a first dose of 50 mg followed by one additional dose of 50 mg within one week of the first dose;
(b) a first dose of 100 mg followed by at least one and up to five additional doses within one week of the first dose;
(c) a first dose of 150 mg followed by at least one and up to four additional doses within one week of the first dose;
(d) a first dose of 200 mg followed by at least one and up to three additional doses within 15 days of the first dose;
(e) a first dose of 300 mg followed by one additional dose of 300 mg within one week of the first dose;
(f) a dose of 400 mg;
(g) a loading dose of 600 mg taken over 1-5 days via administration of: (i) 6 100 mg doses, or (ii) 4 150 mg doses, or (iii) 3 200 mg doses; followed by a maintenance dose of 200 mg within 12 days of the first dose of the loading dose; and
(h) a loading dose of 600 mg taken over 1-5 days via administration of: (i) 6 100 mg doses, or (ii) 4 150 mg doses or (iii) 3 200 mg doses; followed by a maintenance dose of 200 mg one week after completion of the loading dose followed by 200 mg once a week for up to 52 weeks.

In other embodiments, the subject in need of treatment or prevention may be administered about 200 mg of the long half-life 8-aminoquinoline daily for a period of four days, wherein said daily dose may be a single dose or divided doses. In some embodiments, e.g., where a subject is being treated for a non-babesia infection, such as borreliosis and/or Lyme disease, the subject may be further administered a dose of the long half-life 8-aminoquinoline of about 200 mg/week for up to 52 weeks, wherein said weekly dose may be a single dose or divided doses. In each embodiment, the long half-life 8-aminoquinoline may be administered to the subject via the sub-lingual, buccal, and/or intravenous routes, or combinations of those routes of administration. In some embodiments, no more than 11,000 mg of the long half-life 8-aminoquinoline is administered to the subject in a twelve-month period.

In some embodiments, the subject is also administered an additional pharmaceutical agent on one or more days of the protocol for treatment or prevention. For example, in some embodiments, the additional pharmaceutical agent comprises one or more of azithromycin, atovaquone, and/or doxycycline administered to the subject. In other embodiments, the methods of treatment or prevention, and/or uses of the long half-life 8-aminoquinoline, may further comprise administering a second or third agent to the human subject selected from one or more of doxycycline, azithromycin-atovaquone, clindamycin-quinine, artesunate, artemether-lumefantrine, and any other agent[s] recommended by the IDSA or CDC for treating a non-viral tick-borne disease.

It should be understood that the references to dosages or dose, as used herein, should be interpreted according to their common usage, e.g., referring to the amount of the active ingredient (e.g., the dose of the long half-life 8-aminoquinoline, or pharmaceutically-acceptable salt thereof), administered to the subject. It should be further understood that the specific embodiments recited herein are exemplary in nature and not intended to limit the full scope of applicants' invention as would be appreciated by one skilled in the filed from applicants' disclosure.

### BRIEF DESCRIPTION OF THE DRAWINGS

The foregoing will be apparent from the following more particular description of example embodiments of the invention, as illustrated in the accompanying drawings.

FIG. 1A and FIG. 1B: Molecular structure of primaquine and tafenoquine depicted together with summary biological data referenced in the text. Tafenoquine has a longer half-life in vivo and is consequently more potent with a broader spectrum of effects against multiple organisms.

### DETAILED DESCRIPTION OF THE INVENTION

### Definitions

All definitions of substituents set forth below are further applicable to the use of the term in conjunction with another substituent. Unless defined otherwise, all technical and scientific terms used herein have the same meaning as commonly understood by one of ordinary skill in the art to which this invention belongs.

As used herein, the singular forms “a,” “and,” and “the” include plural reference unless the context clearly dictates otherwise. Additionally, the term “comprises” is intended to include embodiments where the method, apparatus, composition, etc., consists essentially of and/or consists of the listed steps, components, etc. Similarly, the term “consists essentially of” is intended to include embodiments where the method, apparatus, composition, etc., consists of the listed steps, components, etc.

As used herein, the term “about” refers to a number that differs from the given number by less than 15%. In other embodiments, the term “about” indicates that the number differs from the given number by less than 14%, 13%, 12%, 11%, 10%, 9%, 8%, 7%, 6%, 5%, 4%, 3%, 2%, or 1%.

As used herein, “asymptomatic” refers to a human subject that has no symptoms of a tick-borne disease and may or may not have been bitten by a tick and may or may not be at risk of being bitten by a tick. For example, an asymptomatic person may have been bitten by a tick and be experiencing no symptoms of a tick-borne illness or be a healthy person contemplating travel or recreation that would increase the risk of a tick bite.

As used herein, “G6PD” means Glucose-6-phosphate dehydrogenase and “G6PD deficiency” refers to a subject being deficient in this enzyme. In humans, treatment of a subject who has G6PD deficiency with an 8-aminoquinoline may cause hemolysis, which can be clinically significant in some cases. Generally, the methods described herein are for subjects without G6PD deficiency.

As used herein, “G6PD-normal” refers to human subjects with normal levels of glucose-6-phosphate dehydrogenase. Normal levels of G6PD may be determined by approved laboratory tests using validated methodology known to those skilled in the art.

The human subject may be an adult or a child. As used herein, a “child” refers to a human subject who is between the ages of 1 day to 17 years of age. The term “adult” refers to a human subject who is 18 years of age or older.

As used herein, “loading phase” or “loading dose(s)” or “initial dose(s)” refers to the initial administration of the material and is at least one dose. For example, the loading phase may be once per day for three consecutive days or less prior to administration of less frequent administration of doses.

As used herein, “subsequent dose(s)” refers to doses administered after initial dose(s) and is at least one dose. The subsequent dose(s) may be the same amount or may be a different amount than the initial dose(s). The subsequent dose(s) may be administered in the same time frame or may be administered in a different time frame than the initial dose(s).

As used herein, “maintenance dose(s)” refers to doses administered after initial and subsequent dose(s).

As used herein, “per day” means in a given 24-hour period.

As used herein, “per week” means in a given 7-day period.

“Three times a day dosing” or “three times per day,” as used herein, refers to three administrations of a composition per every 24-hour period.

“Four times a day dosing” (QDS) or “four times per day,” as used herein, refers to four administrations of a composition per every 24-hour period.

In particular, embodiments of the methods and compositions may use a pharmaceutical composition comprising a compound of Formula (I), or a pharmaceutically acceptable salt thereof. The disclosed compounds of Formula (I), or a pharmaceutically available salt thereof, can be administered to the subject in conjunction with an acceptable pharmaceutical carrier or diluent as part of a pharmaceutical composition for the methods described herein, and according to any of the dosing regimens described herein. Formulation of the compound to be administered will vary according to the route of administration selected (e.g., solution, emulsion, capsule). Suitable pharmaceutical carriers may contain inert ingredients which do not interact with the compound. Standard pharmaceutical formulation techniques can be employed, such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

“Pharmaceutically acceptable carrier” means non-therapeutic components that are of sufficient purity and quality for use in the formulation of a composition of the invention that, when appropriately administered, typically do not produce an adverse reaction, and that are used as a vehicle for a drug substance (e.g., a compound of Formula (I), such as tafenoquine).

The phrase “pharmaceutically acceptable” indicates that the substance or composition must be compatible chemically and/or toxicologically, with the other ingredients comprising a formulation, and/or the mammal being treated therewith.

Pharmaceutical formulations include “pharmaceutically acceptable” and “physiologically acceptable” carriers, diluents or excipients. In this context, the terms “pharmaceutically acceptable” and “physiologically acceptable” include solvents (aqueous or non-aqueous), solutions, emulsions, dispersion media, coatings, isotonic and absorption promoting or delaying agents, compatible with pharmaceutical administration. Such formulations can be contained in a liquid; emulsion, suspension, syrup or elixir, or solid form; tablet (coated or uncoated), capsule (hard or soft), powder, granule, crystal, or microbead. Supplementary compounds (e.g., preservatives, antibacterial, antiviral, and antifungal agents) can also be incorporated into the compositions.

The compounds of the present invention can be formulated into pharmaceutically-acceptable salt forms. Pharmaceutically-acceptable salts of the compounds of the invention can be prepared using conventional techniques. “Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

The phrase “effective amount” means an amount of an agent that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “immunocompromised,” as used herein, refers to anyone with one or more of the following: asplenia, hyposplenism, an autoimmune disease, have been treated with immunosuppressive drugs (for example, a drug from this non-exclusive list: rituximab, cyclophosphamide, vincristine, prednisone, hydrocortisone, and doxyrubicin), or have any pre-existing medical condition(s) that suppress(es) the immune system (for example, a condition from this non-exhaustive list: receipt of an organ transplant, AIDS, B-cell lyphoma, multiple myeloma, and condition associated with a depleted B-cell count, hereditary spherocytosis, Evan syndrome, brain tumor, carcinoma, stomach cancer, and x-linked agammaglobulinemia).

The term “immunocompetent,” as used herein, means anyone who is not immunocompromised as described above.

As used herein, “semi-immune” refers to a resident of a malaria-endemic country who, due to many prior exposures to symptomatic malaria, has developed a partial immunity that usually results in a lack of signs and symptoms of clinical malaria when the presence of malaria parasites in the blood is confirmed by microscopy. Antimalarial drugs work at different doses in semi-immune versus non-immune individuals.

As used herein, “non-immune” refers to individuals who have had insufficient prior exposure to malaria to render them immune to the signs and symptoms of malaria when malaria parasites are confirmed by microscopy to be present. A non-immune individual may also be malaria-naïve if they have never been exposed to malaria before.

As used herein, a subject is “in need of” a treatment if such human or non-human animal subject would benefit biologically, medically, or in quality of life from such treatment (preferably, a human). In some embodiments, the subject has symptoms typical of babesiosis or other non-viral tick-borne diseases and is in need of therapy. In other embodiments, the subject is suspected or known to have been bitten by a tick and is need of post-exposure prophylaxis to prevent contraction of symptomatic disease. In some embodiments, the subject is anticipating traveling or conducting recreational activities (e.g., hiking or camping) that may be associated with a higher risk of being bitten by a tick, and are therefore in need of prophylaxis. In some embodiments, the subject is at increased risk of occupational exposure to tick bites than others in the population (e.g., forestry workers or wilderness guides).

As used herein, the term “inhibit,” “inhibition,” or “inhibiting” refers to the reduction or suppression of a given condition, symptom, or disorder, or disease, or a significant decrease in the baseline activity of a biological activity or process.

As used herein, the terms “subject,” “patient,” and “individual” are used interchangeably and refer to a human of any age or gender.

As used herein, the term “treat,” “treating,” or “treatment” of any disease or disorder refers in one embodiment, to ameliorating the disease or disorder (i.e., slowing or arresting or reducing the development of the disease or at least one of the clinical symptoms thereof). In another embodiment “treat,” “treating,” or “treatment” refers to alleviating or ameliorating at least one physical parameter including those which may not be discernible by the subject. In yet another embodiment, “treat,” “treating,” or “treatment” refers to modulating the disease or disorder, either physically, (e.g., stabilization of a discernible symptom), physiologically, (e.g., stabilization of a physical parameter), or both. In yet another embodiment, “treat,” “treating,” or “treatment” refers to prophylaxis (preventing or delaying the onset or development or progression of the disease or disorder).

As used herein, the term “pre-treat,” “pre-treating,” or “pre-treatment” of any disease or disorder refers to the embodiments described for “treat,” “treating,” or “treatment,” wherein the first administration is prior to exposure (or potential exposure) to said disease or disorder. In particular embodiments, “pre-treat,” “pre-treating,” or “pre-treatment” of any disease or disorder refers to decreasing the severity of said disease or disorder or accelerating the recovery of said disease or disorder, wherein the first administration is prior to exposure (or potential exposure) to said disease or disorder.

As used herein, the term “administration” is intended to include, but is not limited to, the following delivery methods: topical, oral, sub-lingual, buccal, parenteral, subcutaneous, transdermal, transbuccal, intravascular (e.g., intravenous or intra-arterial), intramuscular, subcutaneous, intranasal, and intra-ocular administration. Administration can be local at a particular anatomical site, such as a site of infection, or systemic.

As used herein, “prevent” or “prevention” refers to achieving, partially, substantially, or completely, one or more of the following results: avoiding the disease, disorder, or syndrome resulting from infection by a non-viral tick-borne pathogen; avoiding clinical symptom or indicator associated with a disease, disorder, or syndrome resulting from infection by a non-viral tick-borne pathogen; reducing the severity of the disease, medical complications, disorder, or syndrome resulting from infection by a non-viral tick-borne pathogen; or avoiding death.

As used herein, “symptomatic” refers to a subject in whom symptoms of one or more non-viral tick-borne disease(s) is evident upon clinical evaluation.

As used herein, “tafenoquine” refers to a compound of Formula (I) with the following structure:

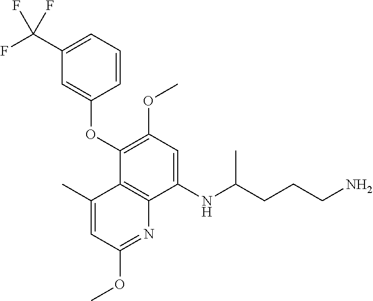 [SMILES: COc1cc(C)c2c(Oc3cccc(C(F)(F)F)c3)c(OC)cc(NC(C)CCCN)c2n1]

which has an alternative name of N (4)-[2,6-Dimethoxy-4-methyl-5-[3-(trifluoromethyl) phenoxy] quinolin-8-yl] pentane-1,4-diamine, or a pharmaceutical acceptable salt thereof. Tafenoquine may also be known as Tafenoquine [INN: BAN], Etaquine, UNII-262P8GS9L9, C24H28F3N303, CHEBI: 172505, AIDS006901, 106635-81-8 (maleate), AIDS-006901, CID115358, SB-252263, WR 238605, WR-238605, WR238605, LS-172012, 1,4-Pentanediamine, N4-(2,6-dimethoxy-4-methyl-5-(3-(trifuluromethyl) phenoxy)-8-quinolinyl-, 106635-80-7, N (4)-(2,6-Dimethoxy-4-methyl-5-((3-trifluromethyle) phenoxy)-8-quinolinyl)-1,4-pentanediamine, N-[2,6-dimethoxy-4-methyl-5-[3-(trifluoromethyl) phenoxy] quinolin-8-yl] diamine, (4-Amino-1-methylbutyl) {2,6-dimethoxy-4-methyl-5-[3-(trifluoromethyl) phenoxy] (8-quinoly)} amine, (R)-N3-(2,6-Dimethoxy-4-methyl-5-(3-trifluoromethyl) phenoxy) quinolin-8-yl) pentane-1,4-diamine, (RS)-N(sup 3)-(2,6-Dimethoxy-4-methyl-5-(3-trifluoro-methylphenoxy) quinolin-8-yl) pentane-1,4-diamine. A pharmaceutically acceptable salt thereof, including,

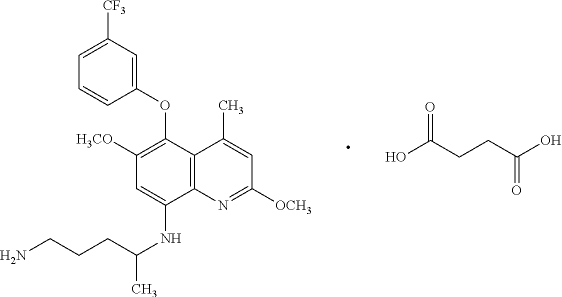 [SMILES: COc1cc(C)c2c(Oc3cccc(C(F)(F)F)c3)c(OC)cc(NC(C)CCCN)c2n1 ; O=C(O)CCC(=O)O]

CAS number for above identified structure of succinate salt 106635-81-8.

The compounds of the invention useful for practicing the methods described herein may possess one or more chiral centers and so exist in a number of stereoisomeric forms. All stereoisomers and mixtures thereof are included in the scope of the present invention. Racemic compounds may either be separated using preparative HPLC and a column with a chiral stationary phase or resolved to yield individual enantiomers utilizing methods known to those skilled in the art. In addition, chiral intermediate compounds may be resolved and used to prepare chiral compounds of the invention.

The compounds described herein may exist in one or more tautomeric forms. All tautomers and mixtures thereof are included in the scope of the present invention. The compounds of the present invention can be administered as the free base or as a pharmaceutically acceptable salt. For example, an acid salt of a compound of the present invention containing an amine or other basic group can be obtained by reacting the compound with a suitable organic or inorganic acid, resulting in pharmaceutically acceptable anionic salt forms. Examples of anionic salts include the acetate, benzenesulfonate, benzoate, bicarbonate, bitartrate, bromide, calcium edetate, camsylate, carbonate, chloride, citrate, dihydrochloride, edetate, edisylate, estotate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate, and triethiodide salts. In one embodiment, the compound of Formula (I) is a hydrochloride salt.

When used herein, a dose range reflected as two numbers means those doses as well as all doses within that range. For example, a dose range from 10 mg-11 mg means 10.0 mg, 10.05 mg, 10.10 mg, 10.15 mg, 10.20 mg, 10.25 mg, 10.30 mg, 10.35 mg, 10.40 mg, 10.45 mg, 10.50 mg, 10.55 mg, 10.60 mg, 10.65 mg, 10.70 mg, 10.75 mg, 10.80 mg, 10.85 mg, 10.90 mg, 10.95 mg, 11.00 mg, as well as any and all amounts therein, such as 10.34 mg, 10.78 mg, etc.

As used herein, “suspected non-viral tick-borne disease” means a subject that has symptoms known to be consistent with a non-viral tick-borne disease. This need not be associated with evidence of a tick bite, because in many cases a tick is not observed attached to a person and/or the timing of a tick bite may not be known. The type of suspected non-viral tick-borne disease will depend on the likely infecting organism. For example, the observation of erythema migrans is likely Lyme disease caused by Borrelia burgdorferi, whereas presentation with fever, malaise, anorexia, and anemia might be suspected to be babesiosis in the absence of another diagnosis.

### Long Half-Life 8-Aminoquinolines

Substitutions at the 2, 4 and 5 positions of the 8-aminoquinoline ring are known to impart a longer half-life and broader spectrum of action against pathogens such as Pneumocystis and Plasmodium. This also seems to be the case for some tick-borne diseases since 4-methyl substituted primaquine is more efficacious than primaquine against Babesia microti (Reubush et al. 1980). Also, one and three doses of tafenoquine were sufficient to clear and cure Babesia in immunocompromised mice and dogs (Liu et al. 2020), whereas daily primaquine administration was only suppressive against Babesia microti in Mongolian jirds, even though these were not reported to be immunocompromised (Ruebush et al. 1980).

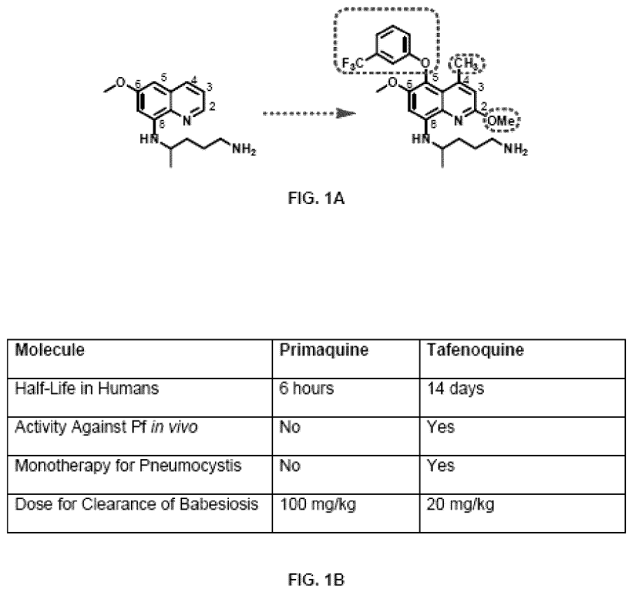

Extension of the half-life of 8-aminoquinolines through substitution at the 2, 4, and 5 positions of the quinoline ring is known to increase the potency and expand the spectrum of action compared to short half-life 8-aminoquinolines as primaquine. See FIG. 1A and FIG. 1B. For example, whereas primaquine has only weak activity against the blood stages of P. falciparum, tafenoquine is quite effective (Baird et al. 2002. Am J Trop Med Hyg. 2002 June; 66 (6): 659-60; Mccarthy et al. 2019. Clin Infect Dis. 2019 Jul. 18; 69 (3): 480-486). Similarly, whilst tafenoquine can be administered as monotherapy to cure Pneumocystis infections in mice, primaquine must be combined with clindamycin to achieve the same outcome (Bartlett et al. ANTIMICROBIAL AGENTS AND CHEMOTHERAPY, February 1991, Vol 35 (2): 277-282). Also, whereas single doses of 20 mg/kg tafenoquine cleared Babesia parasitemia in mice (Mordue and Wormser 2019. 442 jid 2019:220, 1 August), a much higher dose of primaquine is required (100 mg/kg) (Yao et al. 2015. J Infect Dev Ctries. 2015 Sep. 27; 9 (9): 1004-10).

In a thorough QTC study, tafenoquine at doses of up to 1200 mg over three days were not found to increase the upper limit of 90% confidence interval of the QTC interval, thereby meeting the generally accepted regulatory standard for considering a drug to not exhibit a cardiotoxicity liability (Green et al. 2014. J Clin Pharmacol 54:995-1005). Importantly, tafenoquine also did not increase the QTC prolongation known to be associated with chloroquine when the two drugs were coadministered (Green et al. 2014). Tafenoquine may also be safely administered as a loading dose of 600 mg over three days followed by weekly dosing of 200 mg for up to one year without increasing the overall rate of adverse events experienced by patients relative to placebo, with only gastrointestinal effects [mild to moderate] and cornea verticillata being the only specific adverse events for which there was increased risk—these effects are reversible, and not clinically significant [Moreno et al 2021].

### Patient Population

Subjects in need of having their non-viral tick-borne disease prevented or treated include, but are not limited to, the following patient populations: those who are symptomatic ambulatory, those who are symptomatic hospitalized, those who are symptomatic ambulatory or hospitalized who have risk factors for disease progression, which is distinct from immunosuppressed, (in the case of babesiosis age >55 years, fatigue, nausea, diarrhea, duration of symptoms >7 days, lab abnormalities such as depressed/elevated white cell counts, elevated bilirubin, elevated creatinine and parasitemia >10%, or any pre-existing condition or treatment for/or a condition that results in an immunocompromised state, and combinations thereof), those who are symptomatic ambulatory or hospitalized who are immunocompromised (in the case of babesiosis those with asplenia, hyposplenism, an autoimmune disease, who have been treated with immunosuppressive drugs, or who have any pre-existing medical condition that suppresses the immune system), those who are asymptomatic individuals (with or without risk factors) who may be at risk of tick bites, individuals (with or without risk factors) with tick bites who may be at risk of contracting babesiosis or other non-viral tick-borne diseases caused by Babesia and/or Borrelia and/or Anaplasma and/or, Rickettsia and/or Ehrlichia and/or Francisella spp.

### Diagnostic Criteria

For treatment indications of the claimed invention, diagnostic criteria include, but are not limited to: laboratory-confirmed tick-borne disease (such as babeosis) via testing such as blood smear and/or polymerase chain reaction (“PCR”); parasite load (such as parasitemia in blood smear); and suspected infection based upon diagnostic criteria.

### Dosing Regimens

Dosing regimens according to the invention are those that are effective in preventing and/or treating a non-viral tick-borne disease in a given subject. Oral administration and/or formulation are done so as to minimize gastrointestinal (“GI”) upset in the subject, especially when doses ≥400 mg/day are given. Doses above 400 mg of tafenoquine are often not well tolerated (e.g., the dose may cause gastrointestinal issues or toxicity) by adult subjects regardless of the subject's G6PD status. In G6PD normal adult subjects, doses of up to 400 mg of tafenoquine may be well tolerated, while in G6PD deficient subjects, doses of 300 mg or more may not be well tolerated. GI upset may be minimized and/or obviated and/or alleviated by buccal administration, sublingual administration, intravascular (e.g., intravenous or intra-arterial) administration, and/or by using a delivery design (tablet, sheet, etc.) that minimizes GI upset. Maintenance dosing may continue as necessary for six months to one year, provided the total dose administered does not exceed 11,000 mg over a 12-month period.

In particular embodiments, the long half-life 8-aminoquinoline is tafenoquine, or a pharmaceutically acceptable salt thereof. The long half-life 8-aminoquinoline may be administered to the human subject as at least one initial (loading) dose. In particular embodiments, 50 mg-400 mg dose(s) are administered.

In further embodiments, the methods of treatment of a non-viral tick-borne disease and/or methods of prevention of a non-viral tick-borne disease, further comprise administering a second agent, such as a drug, to the human subject. In other embodiments, the method comprises administering to said subject an effective amount of a second agent. In further embodiment, the administration of tafenoquine or said compound of Formula (I) and administration of said second agent is concurrent. In yet other embodiments, the administration of tafenoquine or said compound of Formula (I) and administration of said second agent is not concurrent. In even further embodiments, no second agent is administered.

In other embodiments, the second agent(s) is/are selected from one or more of the following: artemether, artesunate, atovaquone, atovaquone-proguanil, azithromycin, clindamycin, doxycycline, lumefantrine, quinine, and tetracycline.

An embodiment of the invention is a dosing regimen according to Table 1, with or without a second agent. In particular, both methods of treatment of a non-viral tick-borne disease and methods of prevention of a non-viral tick-borne disease, the method comprises administering to said subject an effective amount of tafenoquine or a compound of Formula (I), a pharmaceutically-acceptable salt thereof, or a pharmaceutical composition comprising tafenoquine or a compound of Formula (I),

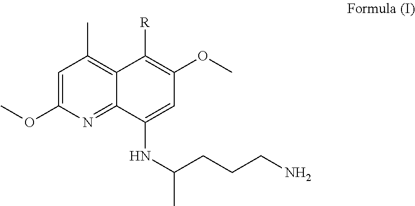

wherein R is any halogen-containing substituent of molecular weight≤205, wherein the administering is in accordance with a dosing regimen according to Table 1.

TABLE 1

| SINGLE DOSE OR | | |
|---|---|---|
| INITIAL DOSE | SUBSEQUENT | MAINTENANCE |
| In REGIMEN | DOSE[s] | DOSE |
| 50 mg | One additional dose | N/A |
| | within one week of | |
| | first dose | |
| 100 mg | At least one, and up to | N/A |
| | five doses within one | |
| | week of first dose | |
| 150 mg | At least one, and up to | N/A |
| | 4 additional doses within | |
| | one week of first dose | |
| 200 mg | At least one, and up to | N/A |
| | three additional doses | |
| | within 15 days of the | |
| | first dose | |
| 300 mg | One additional dose | N/A |
| | within one week of | |
| | first dose | |
| 400 mg | N/A | N/A |

| | |
|---|---|
| LOADING DOSE - 600 mg taken over 1-5 days | 200 mg within 12 |
| via administration of 6 × 100 mg or 4 × 150 mg | days of the first |
| [covering all administration options including as | loading dose |
| outlined in the ARAKODA prescribing information] | |
| LOADING DOSE - 600 mg taken over 1-5 days | 200 mg one week |
| via administration of 6 × 100 mg or 4 × 150 mg | later then 200 mg |
| [covering all administration options including as | once per week |
| outlined in the ARAKODA prescribing information] | thereafter for |
| | up to 12 months |

### EXEMPLIFIED EMBODIMENTS

Embodiment 1—A method for treating or preventing a non-viral tick-borne disease, or a symptom thereof, in a human subject, said method comprising administering an effective amount of a long half-life 8-aminoquinoline to a subject in 10 need thereof.

Embodiment 2—The method of Embodiment 1, wherein organism transmitted by a tick is caused by a Babesia, Rickettsia, Francisella, Anaplasma, Ehrlichia, or Borrelia spp and the disease caused may be one or more of, but is not limited to, the following: African tick bite fever, anaplasmosis, babesiosis, borreliosis, ehrlichiosis, Lyme disease, Mediterranean spotted fever, relapsing tick-fever, Rickettsia parkeri rickettsiosis, rickettsiosis, Rocky Mountain spotted fever, Southern tick-associated rash illness, tickborne relapsing fever, tularemia, and 364D rickettsiosis.

Embodiment 3—The method of Embodiment 1 or 2, wherein the long half-life 8-aminoquinoline is capable of treating or preventing the non-viral tick-borne disease at a minimum single dose or first dose in a regimen of 50 mg.

Embodiment 4—The method of any one of Embodiments 1-3, wherein the long half-life 8-aminoquinoline is tafenoquine or a pharmaceutically acceptable salt thereof.

Embodiment 5—The method of any one of Embodiments 1-4, wherein administration is according to one of the following regimens: (a) a first dose of 50 mg followed by one additional dose of 50 mg within one week of the first dose; (b) a first dose of 100 mg followed by at least one and up to five additional doses within one week of the first dose; (c) a first dose of 150 mg followed by at least one and up to four additional doses within one week of the first dose; (d) a first dose of 200 mg followed by at least one and up to three additional doses within 15 days of the first dose; (e) a first dose of 300 mg followed by one additional dose of 300 mg within one week of the first dose; (f) a dose of 400 mg; (g) a loading dose of 600 mg taken over 1-5 days via administration of: (i) 6 100 mg doses, or (ii) 4 150 mg doses, or (iii) 3 200 mg doses; followed by a maintenance dose of 200 mg within 12 days of the first dose of the loading dose; and (h) a loading dose of 600 mg taken over 1-5 days via administration of: (i) 6 100 mg doses, or (ii) 4 150 mg doses or (iii) 3 200 mg doses; followed by a maintenance dose of 200 mg one week after completion of the loading dose followed by 200 mg once a week for up to 52 weeks.

Embodiment 6—The method of any one of Embodiments 1-5, wherein said subject in need thereof is: symptomatic ambulatory, symptomatic ambulatory with risk factors for disease progression, symptomatic ambulatory and immunocompromised, symptomatic hospitalized, symptomatic hospitalized with risk factors for disease progression, symptomatic hospitalized and immunocompromised, asymptomatic, asymptomatic without risk factors, asymptomatic with risk factors, asymptomatic at risk of tick bite, asymptomatic without risk factors at risk of tick bite, asymptomatic with risk factors at risk of tick bite, one without risk factors who has been bitten by a tick and is at risk of contracting said tick-borne disease, and one with risk factors who has been bitten by a tick and is at risk of contracting said non-viral tick-borne disease.

Embodiment 7—A method for treating or preventing a non-viral tick-borne disease, or a symptom thereof, in a human subject, said method comprising:
(a) administering to said subject an effective amount of a compound of Formula (I), a pharmaceutically-acceptable salt thereof, or a pharmaceutical composition comprising a compound of Formula (I),

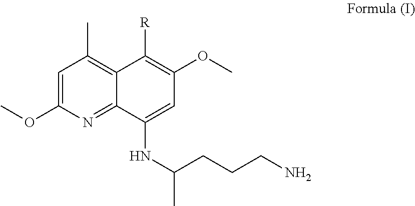

wherein R is any halogen-containing substituent of molecular weight≤205.

Embodiment 8—The method of Embodiment 7, wherein organism transmitted by a tick is caused by a Babesia, Rickettsia, Francisella, Anaplasma, Ehrlichia or Borrelia spp and the disease caused may be one or more of, but is not limited to the following: African tick bite fever, anaplasmosis, babesiosis, borreliosis, ehrlichiosis, lyme disease, Mediterranean spotted fever, relapsing tick-fever, Rickettsia parkeri rickettsiosis, rickettsiosis, Rocky Mountain spotted fever, Southern tick-associated rash illness, tickborne relapsing fever, tularemia, and 364D rickettsiosis.

Embodiment 9—The method of Embodiment 7 or 8, wherein the long half-life 8-aminoquinoline is capable of treating or preventing the non-viral tick-borne disease at a minimum single dose or first dose in a regimen of 50 mg.

Embodiment 10—The method of any one of Embodiments 7-9, wherein the long half-life 8-aminoquinoline is tafenoquine or a pharmaceutically acceptable salt thereof.

Embodiment 11—The method of any one of Embodiments 7-10, wherein administration is according to one of the following regimens: (a) a first dose of 50 mg followed by one additional dose of 50 mg within one week of the first dose; (b) a first dose of 100 mg followed by at least one and up to five additional doses within one week of the first dose; (c) a first dose of 150 mg followed by at least one and up to four additional doses within one week of the first dose; (d) a first dose of 200 mg followed by at least one and up to three additional doses within 15 days of the first dose; (e) a first dose of 300 mg followed by one additional dose of 300 mg within one week of the first dose; (f) a dose of 400 mg; (g) a loading dose of 600 mg taken over 1-5 days via administration of: (i) 6 100 mg doses, or (ii) 4 150 mg doses, or (iii) 3 200 mg doses; followed by a maintenance dose of 200 mg within 12 days of the first dose of the loading dose; and (h) a loading dose of 600 mg taken over 1-5 days via administration of: (i) 6 100 mg doses, or (ii) 4 150 mg doses or (iii) 3 200 mg doses; followed by a maintenance dose of 200 mg one week after completion of the loading dose followed by 200 mg once a week for up to 52 weeks.

Embodiment 12—The method according to any one of Embodiments 7-11, wherein said subject in need thereof is: symptomatic ambulatory, symptomatic ambulatory with risk factors for disease progression, symptomatic ambulatory and immunocompromised, symptomatic hospitalized, symptomatic hospitalized with risk factors for disease progression, symptomatic hospitalized and immunocompromised, asymptomatic, asymptomatic without risk factors, asymptomatic with risk factors, asymptomatic at risk of tick bite, asymptomatic without risk factors at risk of tick bite, asymptomatic with risk factors at risk of tick bite, one without risk factors who has been bitten by a tick and is at risk of contracting said non-viral tick-borne disease, and one with risk factors who has been bitten by a tick and is at risk of contracting said non-viral tick-borne disease.

Embodiment 13—A method for treating or preventing a non-viral tick-borne disease, or a symptom thereof, in a human subject, said method comprising administering tafenoquine to a subject in need thereof.

Embodiment 14—The method according to Embodiment 13, wherein organism transmitted by a tick is caused by a Babesia, Rickettsia, Francisella, Anaplasma, Ehrlichia or Borellia spp and the disease caused may be one or more of, but is not limited to the following: African tick bite fever, anaplasmosis, babesiosis, borreliosis, ehrlichiosis, lyme disease, Mediterranean spotted fever, relapsing tick-fever, Rickettsia parkeri rickettsiosis, rickettsiosis, Rocky Mountain spotted fever, Southern tick-associated rash illness, tickborne relapsing fever, tularemia, and 364D rickettsiosis.

Embodiment 15—The method according to Embodiment 13 or 14, wherein the long half-life 8-aminoquinoline is capable of treating or preventing the non-viral tick-borne disease at a minimum single dose or first dose in a regimen of 50 mg.

Embodiment 16—The method according to any one of Embodiments 13-15, wherein the long half-life 8-aminoquinoline is tafenoquine or a pharmaceutically acceptable salt thereof.

Embodiment 17—The method according to any one of Embodiments 13-16, wherein administration is according to one of the following regimens: (a) a first dose of 50 mg followed by one additional dose of 50 mg within one week of the first dose; (b) a first dose of 100 mg followed by at least one and up to five additional doses within one week of the first dose; (c) a first dose of 150 mg followed by at least one and up to four additional doses within one week of the first dose; (d) a first dose of 200 mg followed by at least one and up to three additional doses within 15 days of the first dose; (e) a first dose of 300 mg followed by one additional dose of 300 mg within one week of the first dose; (f) a dose of 400 mg; (g) a loading dose of 600 mg taken over 1-5 days via administration of: (i) 6 100 mg doses, or (ii) 4 150 mg doses, or (iii) 3 200 mg doses; followed by a maintenance dose of 200 mg within 12 days of the first dose of the loading dose; and (h) a loading dose of 600 mg taken over 1-5 days via administration of: (i) 6 100 mg doses, or (ii) 4 150 mg doses or (iii) 3 200 mg doses; followed by a maintenance dose of 200 mg one week after completion of the loading dose followed by 200 mg once a week for up to 52 weeks.

Embodiment 18—The method according to any one of Embodiments 13-17, wherein said subject in need thereof is: symptomatic ambulatory, symptomatic ambulatory with risk factors for disease progression, symptomatic ambulatory and immunocompromised, symptomatic hospitalized, symptomatic hospitalized with risk factors for disease progression, symptomatic hospitalized and immunocompromised, asymptomatic, asymptomatic without risk factors, asymptomatic with risk factors, asymptomatic at risk of tick bite, asymptomatic without risk factors at risk of tick bite, asymptomatic with risk factors at risk of tick bite, one without risk factors who has been bitten by a tick and is at risk of contracting said non-viral tick-borne disease, and one with risk factors who has been bitten by a tick and is at risk of contracting said non-viral tick-borne disease.

Embodiment 19—The method according to any one of Embodiments 1-18, further comprising administering a second and/or third agent, for example doxycycline, azithromycin-atovaquone, clindamycine-quinine, artesunate, artemether-lumefantrine, or any other agent[s] recommended by the IDSA or CDC, for treating or preventing a non-viral tick-borne disease.

Embodiment 20—The method according to Embodiment 6, Embodiment 12, or Embodiment 18, wherein said risk factors for disease progression are selected from the group consisting of age >55 years, fatigue, nausea, diarrhea, duration of symptoms >7 days, lab abnormalities such as depressed/elevated white cell counts, elevated bilirubin and elevated createnine or any pre-existing condition or treatment for/or a condition that results in an immunocompromised state, and combinations thereof.

Embodiment 21—The method according to Embodiment 6, Embodiment 12, or Embodiment 18, wherein said immunocompromised state results from a condition selected from the group consisting of asplenia, hyposplenism, prior treatment with an immunosuppressive drug, pre-existing auto-immune disease or other condition known to suppress the immune system, and combinations thereof.

Embodiment 22—The method according to Embodiment 6, Embodiment 12, or Embodiment 18, wherein said causative agent is a species of Babesia or the disease is babesiosis.

Embodiment 23—The method according to Embodiment 6, Embodiment 12, or Embodiment 18, wherein said causative agent is a species of Borrelia or the disease is borreliosis or lyme disease.

Embodiment 24—The method according to Embodiment 6, Embodiment 12, or Embodiment 18, wherein said causative agent is a species of Rickettsia or the disease is ricketsiosis.

Embodiment 25—The method according to Embodiment 6, Embodiment 12, or Embodiment 18, wherein said causative agent is a species of Anaplasma or Ehrlichia and the disease is anaplasmosis or ehrlichiosis.

Embodiment 26—The method according to Embodiment 6, Embodiment 12, or Embodiment 18, wherein said causative agent is a species of Francisella and the disease is tularemia.

Embodiment 27—The method according to any one of Embodiments 1-26, wherein said administration is via sub-lingual and/or buccal and/or intravenous route(s).

Embodiment 28—The method according to any one of Embodiments 1-27, wherein said administration is conducted according to the dosing regimen of Table 1 and/or according to any of the Examples.

Embodiment 29—The method according to any one of Embodiments 1-28, wherein no more than 11,000 mg is administered to said subject in a twelve-month period.

Embodiment 30-A kit comprising: (a) a means for testing for a non-viral tick-borne disease and/or symptoms thereof and/or glucose-6-dehydrogenase (G6PD deficiency); (b) a compound of Formula (I), a pharmaceutically-acceptable salt thereof, or a pharmaceutical composition comprising a compound of Formula (I); and

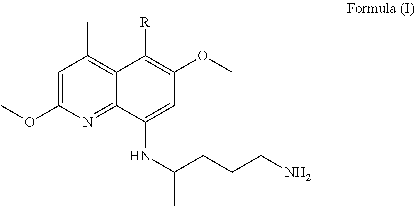

(c) instructions for use, wherein R is any halogen-containing substituent of molecular weight≤205.

Embodiment 31-A kit comprising: (a) a means for testing for a non-viral tick-borne disease and/or symptoms thereof and/or glucose-6-dehydrogenase (G6PD deficiency); (b) a long half-life 8-aminoquinoline; and (c) instructions for use.

Embodiment 32-A kit comprising: (a) a means for testing for a non-viral tick-borne disease and/or symptoms thereof and/or glucose-6-dehydrogenase (G6PD deficiency); (b) tafenoquine; and (c) instructions for use.

Embodiment 33—A method for pre-treating a non-viral tick-borne disease and/or symptoms thereof in a human subject, said method comprising administering to said subject an effective amount of a compound of Formula (I), a pharmaceutically-acceptable salt thereof, or a pharmaceutical composition comprising a compound of Formula (I)

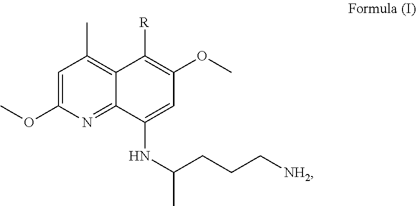

wherein R is any halogen-containing substituent of molecular weight≤205.

Embodiment 34—The method according to Embodiment 33, wherein said Formula (I) compound is tafenoquine or a salt thereof.

### EXAMPLES

### Example 1—Substituted 8-Aminoquinolines are More Active and have Broader Spectrum of Activity than Primaquine

Tafenoquine is more potent and has a broader spectrum of activity against malaria parasites and other pathogens as a consequence of its longer half-life (14 days versus approximately 6 hours). This occurs as a consequence of substitutions at the 2, 4, and 5 positions that increase steric bulk, lipophilicity, and block sites of metabolic attack. Additionally, Ruebush et al. (1980) showed that addition of a methyl group at the 4-position of primaquine, a substitution also present in tafenoquine, exhibits greater efficacy than primaquine in animals against Babesia microti. Tafenoquine and similarly substituted 8-aminoquinolines, such as those illustrated in FIG. 1A and FIG. 2, exhibit more potent and broader spectrums of action against tick-borne pathogens in a manner similar to what is described in the examples for tafenoquine.

### Example 2—Tafenoquine Clears all Blood Stage Parasites in an Active Malaria Infection

Fukuda et al. evaluated tafenoquine v standard of care in Plasmodium vivax malaria patients. Symptomatic P. vivax malaria patients were administered tafenoquine monotherapy (400 mg/day for three days) or standard of care (primaquine+chloroquine). In the tafenoquine arm, all patients cleared their blood stage patients by Day 8, and, for patients for whom data was available, there were no relapses (which can be reinitiated by blood or liver stages and in P. vivax infections are indistinguishable) during the follow up period. This means that tafenoquine monotherapy (1200 mg over three days) is sufficient to eliminate all blood stage organisms during treatment of symptomatic infection.

### Example 3—Tafenoquine Completely Suppresses Development of Blood Stage Parasites when Administered at a Dose of 800 mg Prior to Establishment of Infection in Non-Immunes

McCarthy et al. evaluated the effect of a 600 mg loading dose followed by a 200 mg dose (later approved, with the additional of weekly 200 mg maintenance doses, by FDA, for malaria prevention) on clearance of blood stage parasites of Plasmodium falciparum in non-immune volunteers in a human challenge study. The parasite load was administered three days after the last dose of 200 mg tafenoquine. All control subjects subsequently developed blood stage parasitemia, but none of the tafenoquine subjects did. These data show that a total dose of 800 mg and/or the first four 200 mg doses of the FDA-approved regimen for malaria prophylaxis prevent the development of symptomatic malaria when administered prior to the emergence of symptoms in non-immune subjects.

### Example 4—Low Single Doses of Tafenoquine Clear P. falciparum Gametocytes in Semi-Immune (i.e., Immunocompetent) Individuals

Stone et al. demonstrated that in patients with asymptomatic P. falciparum infections, a single dose of tafenoquine of 1.66 mg/kg when combined with dihydroartemisinin/piperaquine to clear asexual blood stages, rendered gametocytes non-infectious to mosquitoes within seven days. With dihydroartemsinin-piperaquine alone this took 14 days. Therefore, doses as low as 1.66 mg/kg tafenoquine (83 mg in a 50 kg adult or adolescent) cleared gametocytes individuals semi-immune to malaria (i.e., immunocompetent).

### Example 5—Low Single Doses of Tafenoquine Clear P. falciparum Gametocytes in Semi-Immune (Immunocompetent) Individuals

Stone et al. demonstrated that in patients with asymptomatic P. falciparum infections, a single dose of primaquine when combined with dihydroartemisinin/piperaquine to clear asexual blood stages, rendered gametocytes non-infectious to mosquitoes at a level statistically lower than reference values by two days post-dosing. With dihydroartemsinin-piperaquine alone this took between 14 and 21 days. Therefore, single low doses of primaquine, the 8-aminoquinoline progenitor of tafenoquine, cleared gametocytes in individuals semi-immune to malaria (immunocompetent).

### Example 6—Mechanism of Action of Primaquine Against P. falciparum Gametocytes is Via Site Specific Induction of Oxidative Stress Mediated Via Excess Production of Hydrogen Peroxide

Cammarda et al. showed that what the gametocytes of P. falciparum are exposed to primaquine, they are killed by site specific (in bone marrow) excess production of hydrogen peroxide and subsequent oxidative damage. The excess hydrogen peroxide is produced as a result of a two-step biochemical relay whereby (i) sequential oxidation of primaquine to hydroxylated metabolites is mediated by cytochrome-reductase-cytochtome P-450 2D6 complex and (ii) hydroxylated metabolites oxidize spontaneously to quinoneimmine metabolites which are then reduced back to the hydroxylated species generating hydrogen peroxide. The hydrogen peroxide kills parasites locally at sites of such metabolism (bone marrow, liver, elsewhere).

### Example 7—Mechanism of Action of Tafenoquine Against Babesia Parasites is Via Induction of Oxidative Stress

Liu et al. showed that hydrogen peroxide (100 microM) for 2h kills Babesia parasites in vitro by inducing the formation of large vacuoles. The same authors demonstrated that after a single dose of 20 mg/kg in mice, tafenoquine induces the formation of large vacuoles in B. rodhaini blood stages which are visually and quantitatively (i.e., in terms of size) indistinguishable from those produced by hydrogen peroxide in vitro. Antioxidant enzymes were also upregulated in B. rodhaini following single dose treatment with tafenoquine in mice. Therefore, tafenoquine kills Babesia parasites through induction of oxidative stress in a manner analogous to hydrogen peroxide and the effect of primaquine on P. falciparum gametocytes. See Example 6. Trypanosome brucei blood stage forms were also found by Wilkinson et al. (2003) to be killed by exposure to approximately 140 microM hydrogen peroxide for 90 minutes which is also vulnerable to tafenoquine.

These observations indicate that if hydrogen peroxide at microM concentrations kill an organism after a few hours of exposure, it may be susceptible to the effective of tafenoquine in vivo under the right conditions.

### Example 8—Susceptibility of Organisms to Tafenoquine is Related to Intrinsic Susceptibility to the Drug, not Whether or not the Organism Leads an Obligate Intracellular Life Cycle in Mammalian Cells

Tafenoquine kills the extracellular blood stage forms of Trypanosoma brucei and the intracellular amastigote forms of Leishmania major with a similar degree of potency (<4 microM, Yardley et al. 2010; Carvahlo et al. 2015), and Dow U.S. Patent Application Publication No. 2021-0267963 disclosed that tafenoquine was much less active against free-living gram-negative bacteria than free-living gram-positive bacteria. Therefore, the susceptibility of an organism to tafenoquine is not dictated by whether or not an organism has an obligate intracellular life cycle in mammalian cells. Thus, both obligate intracellular bacteria (Rickettsia, Anaplasma, Ehrlichia, Babesia spp) and bacteria thought to exhibit mostly an extracellular life cycle (Borrelia spp) may be susceptible to tafenoquine.

### Example 9—Borrelia Spp and Other Spirochetes are Vulnerable to Hydrogen Peroxide

Although Borrelia spp are gram-negative organisms and therefore might expected to be resistant to tafenoquine based on prior studies (Dow U.S. Patent Application Publication No. 2021-0267963), some, but not all, strains of Borrelia burgdoferi are vulnerable to hydrogen peroxide and that vulnerability may be enhanced under certain physiological conditions. For example, Ramsey et al. 2017 showed that the growth of the B31 5A18NP1 strain was inhibited by hydrogen peroxide at concentrations as low as 62.5 microM for 4 h, Sambri et al. 1991 showed enhanced iron absorption made Borrelia burgdorferi more vulnerable to hydrogen peroxide, and Showman et al. 2016 showed strains with mutations in oxidative response genes that confer greater susceptibility to hydrogen peroxide are also less infectious to the mammalian host.

Steiner et al. 1984 showed that the growth of the spirochete Treponema palludum is significantly inhibited by hydrogen peroxide at concentrations of 50 or 100 microM for two hours in a 3% oxygen atmosphere. Murgia et al. showed that various serovars of Leptospira were killed by exposure to exogenous hydrogen peroxide concentrations <180 microM for as little as 30 min.

These data suggest that spirochetes, despite being gram-native organisms are susceptible to a tafenoquine-like mode of action under certain conditions. It is anticipated that coadministration of standard of care regimens such as doxycycline would enhance the vulnerability of rick-borne spirochetes to tafenoquine.

### Example 10—Tafenoquine is Active Against Babesia Spp in Animal Models

Liu et al. showed that treatment of B. rodhaini-infected Balb/c (immunocompetent) with a single dose of 20 mg/kg tafenoquine cleared parasitemia and resulted in 100% survival, whereas infection was uniformly lethal in untreated animals. In immunocompromised SCID mice a single dose of 20 mg/kg tafenoquine cleared initial parasitemia but did not prevent relapses-death was therefore delayed not prevent as it was in the immunocompetent Balb/c mice. In splenectomized B. gibsoni-infected dogs, parasitemia was cleared and death prevented following administration of a single 100 mg ARAKODA tablet (9-11.5 mg/kg) on Days 12, 19 and 48. Symptoms of infections such as anorexia, elevated respiration and pulse were also alleviated in dogs administered tafenoquine. An equivalent human dosing regimen, adjusting for body surface areas would be approximately 4-6 mg/kg/day x 3 which is 280-420 mg/day for three days in a 70 kg person.

### Example 11—Babesia was Cured by the Prophylactic Regimen of Tafenoquine in a Patient that Failed all Other Treatment. Patient Considered Immunocompetent at the Time

A patient hospitalized with B. microti infection, suspected to be due to immunosuppression induced by prior administration of rituximab, relapsed twice following sequential regimens of azithromycin+atovaquone, a third time after a follow-on regimen of azithromycin+atovaquone+clindamycin, and then a fourth round of chemotherapy with high dose Malarone+atovaquone+clindamycin+azithromycin was suspended due to adverse events. The approved dose of tafenoquine for malaria prevention was then given as monotherapy for six weeks and the patient was cured. This case example demonstrates the potential utility of the FDA-approved regimen of tafenoquine for treatment of babesiosis in humans.

### Example 12—Effectiveness of Antimalarials in Non-Immunes Cannot be Predicted Based on What Doses Work Semi-Immune

Given the complexity, cost and risk of drug development of new medications, it is important, where possible, for the regimen of a drug to be effective both in immunocompetent and immunocompromised individuals.

In the case of malaria, semi-immune individuals develop a resistance to the symptoms of malaria and usually do not develop clinical malaria. However, such semi-immune individuals can still become infected by the malaria-causing parasite, which may be observed in blood sample by microscopy. Whereas non-immune malaria individuals have had insufficient prior exposure to malaria to render them immune to the signs and symptoms of malaria when malaria parasites are confirmed by microscopy to be present. Non-immune malaria individuals may also be malaria-naive if they have never been exposed to malaria before.

For malaria drugs, it is not possible to predict that a drug will have useful prophylactic efficacy in non-immune or malaria-naïve subjects based on data from clinical studies involving semi-immune individuals. Azithromycin displayed worse activity in non-immune/malaria-naïve subjects than semi-immunes, and at a level of efficacy that was much lower than the standard of care to justify further development of azithromycin (azithromycin is not FDA-approved for malaria prophylaxis even 20 years after the clinical work was done.) The table below highlights the results of two studies of azithromycin. Andersen et al., Successful double-blinded, randomized, placebo-controlled field trial of azithromycin and doxycycline as prophylaxis for malaria in western Kenya 26(1) CUN INFECT Dis (1998) 146-50 and Taylor et al., Malaria prophylaxis using azithromycin: a double-blind, placebo-controlled trial in/rain Jaya, Indonesia 28(1) CUN INFECT Dis (1999) 74-81. These data show that for some drugs even at a dose known to be safe and effective for other indications, it is not possible to predict useful prophylactic efficacy against P. falciparum (one of the parasites transmitted by mosquitoes that cause malaria) in malaria-naïve subjects.

TABLE 2
Protective efficacy of azithromycin against P. falciparum
in populations of different immune status

Protective Efficacy (%) Relative
to Placebo Immune Status

| | | PF/Non-immune or |
|---|---|---|
| Regimen | Pf/Semi-Immune | Malaria-naïve |
| Azithromycin | 83% (Anderson) | 72% (Taylor) |
| (250 mg/day) | | |

### Example 13—Tafenoquine is Expected to be Effective for the Treatment of Babesiosis in Ambulatory and Hospitalized Patients Regardless of Immune Status

Patients with babesiosis are treated with either a combination of azithromycin-atovaquone or clindamycin-quinine for 7-10 days. In the majority of those who have competent immune systems, these regimens reduce parasitemia to a level that the patient's immune systems can clear over the course of 12 months (Krause 2008, Krause 2021). In immunocompromised patients, the immune system is unable to clear residual parasitemia, which may result in relapses and the requirement for multiple rounds of antimicrobial therapy and an increased risk of death and complications (Krause 2008).

In patients hospitalized for babesiosis, whom may have a range of immune function from immunocompetent through to severely immunocompromised, the mortality rate is low (1.6%) but the risk of complications such as erythrocyte transfusion (20%), renal failure (20%), respiratory failure (6.8%) and cardiac failure (3.5%) are high (Bloch 2022).

A dose of 800 mg tafenoquine, administered according to any of the dose regimens listed in Table 1 would be expected to exhibit the following clinical benefit in these populations:
Ambulatory Patients: Tafenoquine monotherapy would be anticipated to perform in a manner similar to the standard of care, i.e., in most patients the parasite burden would be largely eliminated by Day 28 no additional rounds of antimicrobial therapy would be required.
Hospitalized Patients: Tafenoquine combined with either of the standard of care regimens would be expected to decrease the risk of complications (such as erythrocyte transfusion, renal failure, respiratory failure and cardiac failure), shorten parasite clearance times relative to the standard of care.
Immunocompromised Patients: Tafenoquine administered in combination with either of the standard of care regimens in immunocompromised patients [irrespective of treatment setting] would be expected to reduce the risk of additional rounds of antimicrobial therapy and hematologic, pulmonary and renal complications.
When combined with artesunate or artemether-lumefantrine, a three-day regimen of tafenoquine (200 mg/day for three days) is expected to provide the same benefits as an 800 mg regimen of tafenoquine without any requirement for coadministration of the usual standard of care.

### Example 14—Tafenoquine is Expected to be Efficacious as Post-Exposure Prophylaxis for Tick-Borne Diseases Following a Tick Bite

Tick bites are a delivery mechanism for delivery of a variety of protozoan and rickesttial spp that cause a variety of diseases with substantial morbidity including lyme disease, babesiosis, erlichiosis and anaplasmosis amongst others. Administration of a single dose of doxycycline within 72h of a tick bite to asymptomatic individuals as post-exposure prophylaxis to prevent lyme disease is an accepted treatment modality. There is no established post exposure prophylaxis for babesiosis or other tick-borne diseases.

Due to common mechanism of action, via a hydrogen peroxide-like effect, across various protozoan, fungal and bacterial spp, and the vulnerability of Babesia spp and spirochetes to hydrogen peroxide, the long half-life, and good safety profile of tafenoquine, it is anticipated that tafenoquine, administered at single or cumulative doses <800 mg outlined in Table 1 would provide the following benefits when utilized as post-exposure prophylaxis following a tick bite:
Monotherapy to reduce the risk of contracting babesiosis.
Monotherapy to reduce the risk of contracting diseases caused by micro-organisms transmitted by ticks that do not cause lyme disease or babesiosis.
Combination therapy with doxycycline to simultaneously prevent lyme+babesiosis and other tick-borne diseases.

### Example 15—FDA Approved Prophylactic for Malaria Will Prevent Babesiosis and Other Tick-Borne Diseases in Individuals at Greater Risk of Exposure to Infected Ticks

The approved dose of tafenoquine for malaria prevention (200 mg/day for three days then 200 mg once per week) for up to six to twelve months is anticipated to reduce the risk of babesiosis, and/or lyme disease, and/or rickettsial diseases and/or other tick-borne diseases when administered prophylactically to individuals at greater risk of contracting a tick bite.

### Example 16—Tafenoquine Anticipated to be Active Alone or in Combination with Standard of Care Regimens in Animal Models

Tafenoquine administered as monotherapy as three doses of 50 mg/kg or less or in combination with standard of care regimens (doxycycline, azithromycin-atovaquone, etc.) is expected to increase survival, decrease micro-organism load, persistence and antibiotic tolerance in appropriate animal models of tick-borne spirochetes (e.g., Borrelia, Rickettsia, Ehrlichia and Anaplasma spp). In the specific case of lyme disease this may occur as a result of activity against both regular extracellular forms and/or intracellular forms in macrophages and endothelial (and likely other) cells.

### Example 17—Human Infections Cause by Tick-Borne Pathogens are Expected to be Susceptible to Tafenoquine at the Same Doses as Outlined in Examples 13

The standard of care treatments for human tick-borne diseases such as lyme disease, Rocky Mountain spotted fevers, and anaplasmosis (amongst others) are imperfect because in a minority of patients radical cure is not achieved, or because the spirochetes naturally persist, or become tolerant to antibiotics. Due to the common mechanism of action of tafenoquine across several pathogenic species, its long half-life and safety profile, it is anticipated that combining standard of care treatments with tafenoquine at the doses listed in Example 13 for the treatment of such diseases in patients with one or more of such infections will reduce in-hospital complications, and/or decreased microorganism clearance times, and/or decreased persistence/antimicrobial tolerance, and/or retreatment rates relative to the standard of care.

The teachings of all patents, published applications, and references cited herein are incorporated by reference in their entirety for all purposes.

While this invention has been particularly shown and described with references to the example embodiments thereof, it will be understood by those skilled in the art that various changes in form and details may be made therein without departing from the scope of the invention encompassed by the appended claims.

### REFERENCES

Bloch et al. 2022. Epidemiology of Hospitalized Patients with Babesiosis, United States, 2010-2016. Emerging Infectious Diseases; 28:354-362.
Cabello et al. 2022. Borreliella burgdorferi Antimicrobial-Tolerant Persistence in Lyme Disease and Posttreatment Lyme Disease Syndromes mBIO: https://doi.org/10.1128/mbio.03440-21.
Cammarda et al. 2019. Antimalarial activity of primaquine operates via a two-step biochemical relay. Nature Communications; 10:3226.
Carvahlo et al. 2015. The oral antimalarial drug tafenoquine shows activity against Trypanosoma brucei. Antimicrob Agents Chemo; 59:6151-6160.
Chapman et al. 2006. Diagnosis and management of tickborne rickettsial diseases: Rocky mountain spotted fever, ehrlichoses, and anaplasmosis-United States. A practical guide for physicians and other health-care and public health professionals. MMWR; 55 (RR04): 1-27.
Fukuda et al. 2017. A randomized, double-blind, active-control trial to evaluate the efficacy and safety of a three-day course of tafenoquine monotherapy for the treatment of Plasmodium vivax malaria. PLOS ONE 12 (11): e0187376.
Krause et al. 2008. Persistent and Relapsing Babesiosis in Immunocompromised Patients. CID; 46:370-6.
Krause et al. 2021. Clinical Practice Guidelines by the Infectious Diseases Society of America (IDSA): 2020 Guideline on Diagnosis and Management of Babesiosis. CID; 72: e49-e64.
Kugeler et al. 2010. A review of death certificates listing Lyme disease as a cause of death in the United States. CID; 52:364-367.
Lantos et al. 2021. Clinical practice guidelines by the Infectious Diseases Society of America (IDSA), American Academy of Neurology (AAN), and American College of Rheumatology (ACR): 2020 Guidelines for the Prevention, Diagnosis and Treatment of Lyme Disease. CID; 72:1-8.
Liu et al. 2021. Tafenoquine is a promising drug candidate for the treatment of babesiosis. Antimicrobial Agents Chemo; 65: e00204-21.
Ma et al. 1991. Intracellular localization of Borrelia burgdorferi within human Endothelial cells. Infection and Immunity; 59:671-678.
Mace et al. 2021. Malaria Surveillance-United States, 2017. MMWR; 70:1-32.
Marcos et al. 2022. Use of tafenoquine to treat a patient with relapsing babesiosis with clinical and molecular evidence of resistance to azithromycin and atovaquone. IDCases; 27: e01460.
McCarthy et al. 2019. Blood schizonticidal activity and safety of tafenoquine when administered as chemoprophylaxis to healthy, non-immune participants followed by blood stage Plasmodium falciparum challenge: A randomized, souble blind, placebo-controlled Phase Ib study. CID; 69:480-486.
Montgomery et al. 1993. The fate of Borrelia burgdorferi, the agent for lyme disease, in mouse macrophages. J Immunol; 150:909-915.
Moreno et al 2021. Long-term safety of the tafenoquine antimalarial chemoprophylaxis regimen: A 12-month, randomized, double-blind, placebo-controlled trial. TMAID 45:102201
Murgia et al. 2002. Leptospires are killed in vitro by both oxygen-dependent and independent reactions. Infection Immunity; 70:7172-7175.
Ramsey et al. A high throughput genetic screen identifies previously uncharacterized Borrelia burgdorferi genes important for resistance against reactive oxygen and nitrogen species. PLOS Pathog; 13 (2): e1006225.
Rizk M A, El-Sayed SAE, Al-Araby M, Igarashi I (2022) Effect of methanolic extract from Capsicum annuum against the multiplication of several Babesia species and Theileria equi on in vitro cultures, and Babesia microti in mice, Veterinary World, 15(1): 76-82.
Ruebush et al. 1980. Chemotherapy of Babesia microti infections in Mongolian Jirds Antimicrob Agents Chemo; 18:289-291.
Sambri et al. 1991. Susceptibility of iron-loaded Borrelia burgdorferi to killing by hydrogen peroxide and human polymorphonuclear leucocytes. FEMS Microbiol Lett; 65:67-71.
Showman et al. 2016. Gene bb0318 is critical for the oxidative stress response and unfectivity of Borrelia burgdorferi. Infection and Immunity; 84:3141-3151.
Steiner et al. 1984. Susceptibility of Treponema pallidum to the toxic products of oxygen reduction and the non-treponemal nature of its catalase. Br J Vener Dis; 60:14-22.
Stone et al. 2022. Pyronaridine-artesunate or dihydroartemisinin-piperaquine combined with single low-dose primaquine to prevent Plasmodium falciparum malaria transmission in Oelessebougou, Mali: a four-arm, single-blind, phase 2/3, randomized trial. Lancet Microbe 2022; 3: e41-e51.
Stone et al. 2022. Single low-dose tafenoquine combined with dihydroartemisinin-piperaquine to reduce Plasmodium falciparum transmission in Ouelessebougou, Male: a phase 2, single blind, randomized clinical trial. Lancet Microbe: https://doi.org/10.1016/S2666-5247 (21) 00356-6.
Wlikinson et al. 2003. RNA interference identifies two hydroperoxide metabolizing enzymes that are essential to the bloodstream form of the African trypanosome. J Biol Chem; 278:31640-31646.
Yardley et al. 2010. Antileishmanial and antitrypanosomal activities of the 8-aminoquinoline tafenoquine. Antimicrob Agents Chemo 54:5356-5358.

1.-28. (canceled)

29. A method for preventing a tick-borne disease, or a symptom thereof, in a human subject, said method comprising
identifying a human subject with potential exposure to a tick-borne pathogen, wherein said tick-borne pathogen is a parasite of a Babesia or Borrelia species; and
administering to said human subject an effective amount of tafenoquine.

30. The method according to claim 29, wherein the tick-borne disease is babesiosis.

31. The method according to claim 30, wherein said subject is co-infected or suspected of being co-infected with a spirochete of a Borrelia species and/or is suffering from borreliosis or Lyme disease.

32. The method according to claim 29, wherein said potential exposure is a known or suspected tick bite.

33. The method according to claim 29, wherein said potential exposure is a known or suspected tick bite from a tick known or suspected to be a blacklegged tick.

34. The method according to claim 29, wherein said potential exposure is a known tick bite from a blacklegged tick.

35. The method according to claim 29, wherein the tafenoquine is administered to the subject according to one of the following regimens:
(a) a first dose of 50 mg followed by one additional dose of 50 mg within one week of the first dose;
(b) a first dose of 100 mg followed by at least one and up to five additional doses within one week of the first dose;
(c) a first dose of 150 mg followed by at least one and up to four additional doses within one week of the first dose;
(d) a first dose of 200 mg followed by at least one and up to three additional doses within 15 days of the first dose;
(e) a first dose of 300 mg followed by one additional dose of 300 mg within one week of the first dose;
(f) a dose of 400 mg;
(g) a loading dose of 600 mg taken over 1-5 days via administration of: (i) 6 100 mg doses, or (ii) 4 150 mg doses, or (iii) 3 200 mg doses; followed by a maintenance dose of 200 mg within 12 days of the first dose of the loading dose; and
(h) a loading dose of 600 mg taken over 1-5 days via administration of: (i) 6 100 mg doses, or (ii) 4 150 mg doses or (iii) 3 200 mg doses;
followed by a maintenance dose of 200 mg one week after completion of the loading dose followed by 200 mg once a week for up to 52 weeks.

36. The method according to claim 29, wherein about 200 mg of the tafenoquine is administered to the subject daily for a period of four days, wherein said daily dose may be a single dose or divided doses.

37. The method according to claim 36, wherein the subject is further administered a dose of about 200 mg/week for up to 52 weeks, wherein said weekly dose may be a single dose or divided doses.

38. The method according to claim 29, wherein the subject is also administered an additional pharmaceutical agent on one or more days of the treatment.

39. The method according to claim 38, wherein said additional pharmaceutical agent comprises one or more of azithromycin, atovaquone, and doxycycline.

40. The method according to claim 29, wherein the tafenoquine is administered to the subject via one or more of sub-lingual, buccal, and intravenous routes.

41.-61. (canceled)

62. A method for preventing a tick-borne disease, or a symptom thereof, in a human subject, said method comprising
identifying a human subject with increased risk of exposure to a tick-borne pathogen, wherein said tick-borne pathogen is a parasite of a Babesia species; and
administering to said human subject an effective amount of tafenoquine.

63. The method according to claim 62, wherein the tick-borne disease is babesiosis.

64. The method according to claim 62, wherein said identifying a subject at increased risk of exposure comprises identifying a subject that is travelling to and/or conducting recreational or occupational activities in an area or environment associated with increased risk of tick bite.

65. The method according to claim 64, wherein said increased risk of tick bite comprises increased risk of a blacklegged tick bite.

66. The method according to claim 62, wherein the tafenoquine is administered to the subject according to one of the following regimens:
(a) a first dose of 50 mg followed by one additional dose of 50 mg within one week of the first dose;
(b) a first dose of 100 mg followed by at least one and up to five additional doses within one week of the first dose;
(c) a first dose of 150 mg followed by at least one and up to four additional doses within one week of the first dose;
(d) a first dose of 200 mg followed by at least one and up to three additional doses within 15 days of the first dose;
(e) a first dose of 300 mg followed by one additional dose of 300 mg within one week of the first dose;
(f) a dose of 400 mg;
(g) a loading dose of 600 mg taken over 1-5 days via administration of: (i) 6 100 mg doses, or (ii) 4 150 mg doses, or (iii) 3 200 mg doses; followed by a maintenance dose of 200 mg within 12 days of the first dose of the loading dose; and
(h) a loading dose of 600 mg taken over 1-5 days via administration of: (i) 6 100 mg doses, or (ii) 4 150 mg doses or (iii) 3 200 mg doses; followed by a maintenance dose of 200 mg one week after completion of the loading dose followed by 200 mg once a week for up to 52 weeks.

67. The method according to claim 62, wherein about 200 mg of tafenoquine is administered to the subject daily for a period of four days, wherein said daily dose may be a single dose or divided doses.

68. (canceled)

69. The method according to claim 62, wherein the tafenoquine is administered to the subject via one or more of sub-lingual, buccal and intravenous routes.

70. (canceled)

71. The method according to claim 62, further comprising administering an additional pharmaceutical agent to the subject selected from one or more of doxycycline, azithromycin-atovaquone, clindamycin-quinine, artesunate, artemether-lumefantrine, and any other agent[s] recommended by the IDSA or CDC, for treating or preventing a non-viral tick-borne disease.

72.-87. (canceled)
